(19) United States
(12) Patent Application Publication
(10) Pub. No.: US 2020/0405528 A1
(43) Pub. Date: Dec. 31, 2020

(54) CLIMAX CONTROL CONDOM

(71) Applicant: Mario Rodolfo Ovalle Aguilera, Guatemala (GT)

(72) Inventor: Mario Rodolfo Ovalle Aguilera, Guatemala (GT)

(21) Appl. No.: 17/019,476

(22) Filed: Sep. 14, 2020

(51) Int. Cl. A61F 6/04; B29C 41/14; B29C 35/02

(52) U.S. Cl. CPC A61F 6/04; B29C 41/14; B29L 2031/7538; A61F 2006/047; B29C 35/02

(57) ABSTRACT

This present patent application discloses a climax control condom made up of latex to reduce loss of sensation while delaying the ejaculation comprising a closed end of a thick layer of latex anatomically made to cover the glans with a rounded pleat to ensure that the condom will stay in place and not slip during intercourse, a sheath body made up of a thin layer of latex to increase the sensation in the underside of the shaft, and an open end having a collar to reduce slipping of the condom during intercourse. The condom is made by the common dipping process which consists of feeding a latex compound into temperature-controlled tanks into which glass formers are dipped. The formers pick up a thin film of latex, and in the case of the closed end two more layers are added. The latex coating is then dried and, after drying, the condom is then passed through an oven to vulcanize the latex.

### BACKGROUND

The present invention relates generally to the field of human male condoms. More specifically, the present invention relates to a novel climax control condom to be worn over a penis to delay men's climax during a sexual activity while maintaining a long-lasting sensation and increased sexual pleasure. The climax control condom is also used for contraceptive and prophylactic use. The climax control condom may be used in vaginal or anal intercourse, to prevent sexually transmitted diseases. The present invention reduces the loss of sensation through the entire penis and its design will reduce slip during intercourse. The climax control condom provides contraception, but also serves to reduce the spread of sexually transmitted diseases. The condom of the present invention has a closed end to cover the glans, a sheath body to cover the sheath, and an open end having a collar forming a three-part condom. Accordingly, the present specification makes specific reference to the presently described invention. However, it is to be appreciated that aspects of the present invention are also equally amenable to other like applications, devices and methods of manufacture.

By way of background, a male condom is a sheath-shaped barrier device worn over a penis and is used during sexual intercourse to reduce the probability of pregnancy or a sexually transmitted infections (STI). The male condom is rolled onto an erect penis before intercourse and works by forming a physical barrier which blocks semen from entering the body of a sexual partner. Male condoms are typically made from latex and, less commonly, from polyurethane, polyisoprene, or lamb intestine.

Conventional male condoms may slip off the penis after ejaculation or break due to improper application or physical damage, thus, reducing the efficiency of the condoms. Further, conventional thick condoms cause a complete loss of sensation for both male and female during sexual intercourse. Thick condoms, although, easier to wear, and there is a lower chance of them breaking or tearing during sex, however causes, loss of sensation due to uniform thickness along the complete length of the condom. The conventional thick condom does not permit adequate sensitivity for the active male during sexual intercourse because it constricts sensation. Also, because the condom is fitted to move along with the penis, it prevents direct tactile contact and friction between the penis of the active partner and the vaginal or rectal wall of the passive partner during sexual intercourse.

To delay climax and prolong excitement for longer-lasting sex, some condoms use suspending agent benzocaine at the reservoir tip of the condom. However, the effect of benzocaine takes sometime such as 5-10 min. to take effect which causes problems and obstruction for both male and female.

Also, men can have sexual dysfunctions or changes in sexual response based on age, illness, medication or other factors that can make it difficult to perform sexually. The problems can be physical, emotional, or psychological, or a combination of such factors. Some solutions include drugs or other chemical or hormonal aid. However, the side effects of drugs are seldom completely understood, and may over the long term exacerbate the problem or lead to other problems. Men have erectile dysfunction problems which render them unable to perform sexually. What is needed is a therapeutic prosthetic device that enables a person to experience the stimulation and sensations of a sexual event.

Thus, people desire a climax control condom that can be used to delay and provide control over the men's culmination point of climax along with reducing the loss of sensation through the entire penis without using any agent such as benzocaine.

Various proposals for an efficient and easy-to-use condom have been made but none of them appear to be commercially available to the consumer that solves the longstanding problems associated with the ability to provide desired sensation while delaying the climax during a sexual activity.

Therefore, there exists a long felt need in the art for a male condom that overcomes problems associated with traditional thick latex condoms. There is also a long felt need in the art for an improved male condom that delays climax but at the same time let the male enjoy the sensation without the chance of getting too excited and ejaculate. Additionally, there is a long felt need in the art for a male condom that does not use a suspending agent benzocaine at the reservoir tip of the condom. There is also a long felt need in the art for a male condom that prevents sexually transmitted diseases. Additionally, there is a long felt need in the art for a male condom product that does not slip during intercourse. Finally, there is a long felt need in the art for an improved male condom product that provides control over the men's culmination point of climax and reduce the loss of sensation through the entire penis.

The subject matter disclosed and claimed herein, in one embodiment thereof, comprises an improved male condom product that has a thick closed end that is anatomically made to cover the glans with a rounded pleat, a thin sheath body to increase the sensation in the underside of the shaft and an open end has a collar to reduce the slipping of the condom during use. The condom is made up of a suitable material such as Latex and may have a length of 8 inches.

The male condom of the present invention is made up by a dipping process which consists of feeding a latex compound into temperature-controlled tanks into which glass formers are dipped. The formers pick up a thin film of latex, and in the case of the closed end two more layers are added.

In this manner, the male condom product of the present invention accomplishes all of the forgoing objectives, and provides a relatively safe, easy, convenient and cost-effective solution to use condom according to the wants and needs of the people for maintain the desired sensation while delaying the ejaculation. The male condom product of the present invention is also user friendly inasmuch as it is less expensive.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

In view of the foregoing, it is an object of the present invention to provide an improved male condom that provides control over the men's culmination point of climax.

It is an object of the present invention to reduce the loss of sensation through the entire penis.

It is an object of the present invention to provide an improved condom that does not slip during intercourse.

In view of the foregoing, it is an object of the present invention to provide an improved condom that delays climax but at the same time maintain the sensation without the chance of getting too excited and ejaculate.

It is an object of the present invention to provide an improved condom for use in vaginal or anal intercourse and that prevents sexually transmitted diseases.

In accordance with the objects of the present invention, an improved condom according to an embodiment of the invention has a thicker layer of a suitable material such as latex to complete cover the head of a penis, with the thicker layer reducing the sensation at the underside of the glans. To allow the male sensation, the part of the condoms that is in contact with the underside of the shaft has a thin layer of latex in order to stimulate the sensitive part of the penis. During use, the improved condom requires to be properly worn over the penis.

The subject matter disclosed and claimed herein, in one embodiment thereof, comprises a disposable latex condom having a closed end of approximate 85 mm length latex layer having a thickness of 0.15 mm and is anatomically made to cover the glans with a rounded pleat to ensure that the condom will stay in place and not slip during intercourse, a sheath body of approximate 110 mm length latex layer having a thickness of 0.05 mm to increase the sensation in the underside of the shaft, an open end having a collar to reduce a possibly slipping during sexual intercourse.

In a further embodiment of the present invention, manufacturing method of an improved condom is disclosed and comprising feeding a latex compound into temperature-controlled tanks into which glass formers are dipped, picking up thin form of latex by glass formers, adding two additional layers of latex to a closed end of the condom, drying the latex coating, and passing the condom through an oven to vulcanize the latex.

In a further embodiment of the present invention, a disposable condom to maintain the male sensation is disclosed and comprising a thin layer of latex to contact with the underside of the shaft to stimulate the penis and a thick layer of latex to cover the glans to reduce the sensation at the underside of the glans.

In yet another embodiment of the present invention, a method of using a disposable condom of the present invention is disclosed to maintain the sensation while delaying the ejaculation. The steps including acquiring a disposable condom product, wearing the condom over an erect penis such that a closed end of the condom covers a glans portion, a sheath body contacts the underside of a shaft to stimulate the penis, and the condom does not slip during the intercourse. The condom of the present invention is particularly advantageous as the innovative design of the condom overcomes the problems associated with traditional thick latex condoms of a complete loss of sensation while delaying the men's climax.

While the foregoing describes the present invention in relation to the drawings and embodiments, it is to be understood that the description is not intended to limit the scope of the invention to the drawings or embodiments described herein. To the contrary, the description is intended to cover all alternative modifications and equivalents that may be included in the spirit and the scope of the invention as defined by the appended claims.

### BRIEF DESCRIPTION OF THE DRAWINGS

The description refers to provided drawings in which similar reference characters refer to similar parts throughout the different views, and in which:

FIG. 1 illustrates an elevation view of climax control condom of the present invention in accordance with the disclosed architecture.

FIG. 2 illustrates a section view climax control condom of the present invention in accordance with the disclosed architecture.

FIG. 3 illustrates a diagrammatic representation of steps of manufacturing of the climax control condom of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is an improved condom according to an embodiment of the invention having a thicker layer of a suitable material such as latex to completely cover the head of a penis, with the thicker layer reducing the sensation at the underside of the glans. To allow the male sensation, the part of the condoms that is in contact with the underside of the shaft has a thin layer of latex in order to stimulate the sensitive part of the penis. During use, the improved condom requires to be properly worn over the penis.

As noted above, there exists a long felt need in the art for a male condom that overcomes problems associated with traditional thick latex condoms. There is also a long felt need in the art for an improved male condom that delays climax but at the same time let the male enjoy the sensation without the chance of getting too excited and ejaculate. Additionally, there is a long felt need in the art for a male condom that does not use a suspending agent benzocaine at the reservoir tip of the condom. There is also a long felt need in the art for a male condom that prevents sexually transmitted diseases. Additionally, there is a long felt need in the art for a male condom product that does not slip during intercourse. Finally, there is a long felt need in the art for an improved male condom product that provides control over the men's culmination point of climax and reduce the loss of sensation through the entire penis.

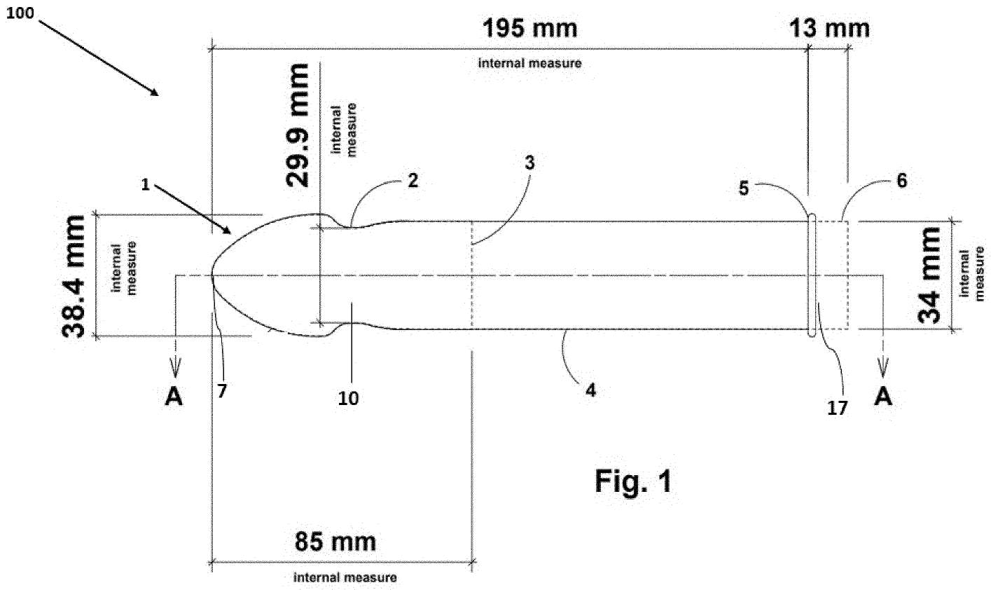

Referring initially to the drawings, FIG. 1 illustrates an elevation view of climax control condom of the present invention in accordance with the disclosed architecture. As shown in FIG. 1, the climax control condom 100 of the present invention has a final length of 195 mm; with an additional length 6 of 13 mm to roll up at an open-end side 17. As it is well known in the art, due to the anatomic design of the condoms there are different internal diameters. The condom 100 having a close end 1 has internal diameter 38.4 mm. Throat section 2 has an internal diameter 10 of 29.9 mm and is designed to reduce the slipping possibility of the sheath body 4 of the condom 100. The sheath body 4 of the condom 100 has an internal diameter of 34 mm. In accordance with the mainly object of the present invention which is to provide men control over the culmination point of the climax, there is a thicker layer of latex that completely covers the glans with a length of 85 mm, starting from a first end 7 at closed end 1 of the condom 100 to a desired point 3. Further, a collar 5 is present at the open end 17 formed by rolled up latex of the condom 100. The collar 5 may be made up of polyurethane, silicone or latex and is placed at the open end 17 of the condom 100.

In alternate embodiments, the final length of the condom 100 can be in the range from 185 mm-205 mm. The close end 1 may have internal diameter in the range 36 mm-40 mm. Further, the throat section 2 may have an internal diameter in the range 25-32 mm.

The advantage of the condom 100 is to improve the performance of people who ejaculate prematurely or earlier than desired while maintaining the sensation by having a thicker layer of latex to cover the glans and a thinner layer of latex to cover the shaft of the penis.

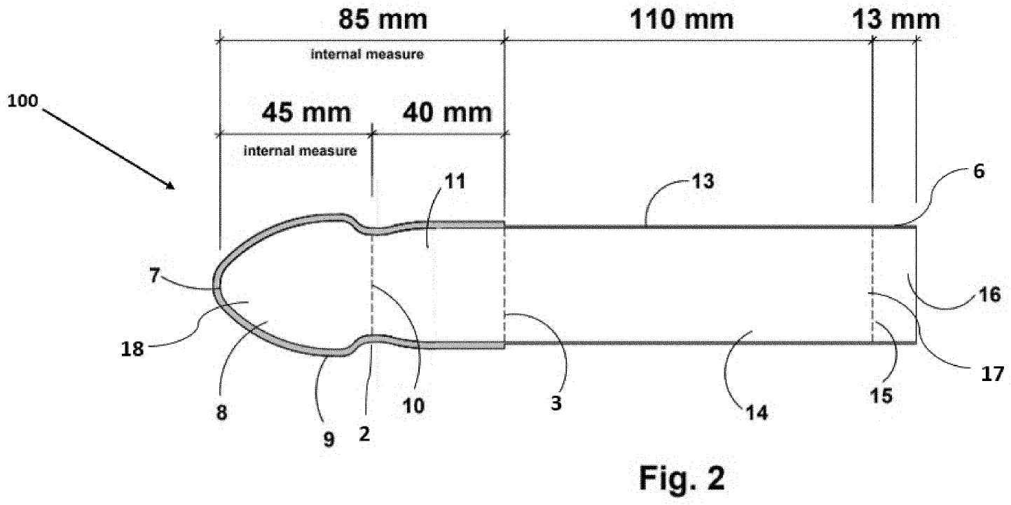

FIG. 2 illustrates a section view of climax control condom of the present invention in accordance with the disclosed architecture. As shown in FIG. 2, the latex layer 9 underside of the glans 8 has a thickness of 0.15 mm extending from a first end 7 to a second end 3 with a length of 85 mm forming a closed end 1. A throat section 2 is defined from a first end 7 having a length of 45 mm and an extension 11 of the thicker layer of latex with a length of 40 mm from throat section 2 to the second end 3 is given for minimal slip during intercourse. In order to allow sensation at the underside of the shaft 14, the sheath body 4 of the condom 100 from point 3 to point 15 is made with a thin layer 13 of latex having thickness of 0.05 mm. From point 15 where the collar 5 is present, there is 13 mm of additional latex 6 present on the condom body 100, extending to an extension point 16. The additional latex 6 length of 13 mm is used to roll up at the open end 17 after the dipping process and before vulcanization process during manufacturing, to form a collar 5.

In alternate embodiments, the thicker layer 9 of the latex of the condom 100 can have thickness of more than 0.15 mm but less than 0.2 mm. The thinner layer 13 of the latex can have thickness more than 0.05 mm but less than 0.08 mm.

As described above, the condom 100 of the invention has thicker layer 9 of latex at a closed end 1 so as to completely cover the head of the penis. The thicker latex layer 9 reduces the sensation at the underside of the glans and thus reduces the chances of ejaculation. The sheath 4 has a thin layer 13 of latex in order to stimulate underside of the shaft of penis to maintain the desired sensation.

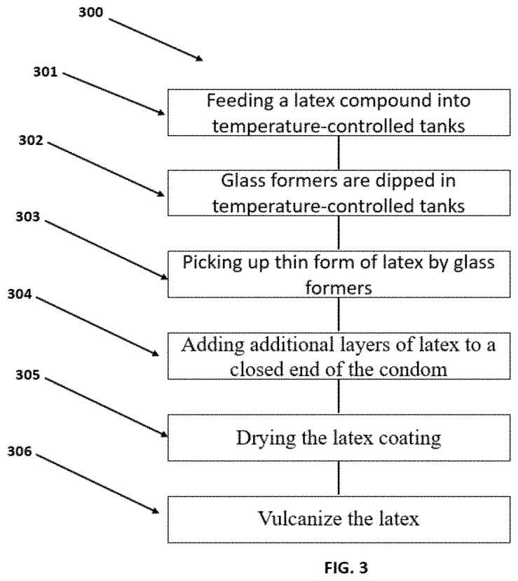

FIG. 3 illustrates a diagrammatic representation of steps of manufacturing of the climax control condom of the present invention in accordance with the disclosed architecture. The manufacturing method 300 of the condom of the present invention is disclosed and comprising an initial step 301 of feeding a latex compound into temperature-controlled tanks, step 302 of dipping the glass formers in temperature-controlled tanks, step 303 of picking up thin form of latex by the glass formers, then the step 304 of adding additional layers of latex to a closed end of the condom 100, step 305 of drying the latex coating, and the final step 306 of passing the condom 100 through an oven to vulcanize the latex. The temperature of the oven can be between 60° C. and 150° C. The condom 100 produced by the process 300 is free from chemical accelerators induced allergies and has better biodegradability.

The present invention advantageously provides climax control condom 100 which, when compared to conventional thick condoms, provides a long-lasting sensation during intercourse, delay climax without the chance of getting too excited and ejaculate. In comparison to the thick condoms, the climax control condom 100 reduces the glans sensation and increases the shaft feeling to provide control over the men's culmination point of climax.

In one embodiment, the condom 100 may have a lubricant coating or spermicide thereon. Further, the condom 100 has sufficient structural rigidity and tensile strength providing protection against the transmission of sexually transmitted diseases as well as protection against unintentional pregnancies, is disposable and can be removed and discarded in the usual fashion after use. The condom 100 may have dots, pleats and the like on the outside of the latex for extra pleasure during the sexual activity. In less preferred embodiment, the condom 100 may be made up of other materials like Polyurethane, Polyisoprene, etc.

The present invention represents an improvement in both the structure and the methods for manufacturing male condoms. The present invention provides an improved condom that less expensive to manufacture while maintaining a high-quality product. The invention overcomes the issues experienced in the prior art that resulted in inefficient and ineffective climax control condoms.

The container to hold condom 100 of the present invention may include, but are not limited to, tubs, flow wrap pouches, individual sachets, chained sachets comprising a tear line between each sachet, and other forms known in the art as suitable for storing nonwoven articles.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “climax control condom”, “improved condom”, “male condom”, and “condom” are interchangeable and refer to the climax control condom product 100 of the present invention.

Notwithstanding the forgoing, the climax control condom 100 of the present invention can be of any suitable size and configuration as is known in the art without affecting the overall concept of the invention, provided that it accomplishes the above stated objectives. One of ordinary skill in the art will appreciate that the size, configuration and material of the climax control condom 100 as shown in the FIGS. are for illustrative purposes only, and that many other sizes of the climax control condom 100 are well within the scope of the present disclosure. Although the dimensions of the climax control condom 100 are important design parameters for user convenience, the climax control condom 100 may be of any size that ensures optimal performance during use and/or that suits user's need and/or preference.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

| | |
|---|---|
| Climax Control Condom | 100 |
| Close end | 1 |
| Throat section | 2 |
| Second end | 3 |
| Sheath body | 4 |
| Collar | 5 |
| Additional length | 6 |
| First end | 7 |
| Glans | 8 |
| Thick latex layer | 9 |
| Internal diameter of throat | 10 |
| Thick layer extension over glans portion | 11 |
| Thin latex layer | 13 |
| Shaft | 14 |
| End point | 15 |
| Extension point | 16 |
| Open end | 17 |
| Climax control condom manufacturing | 300 |
| Feed latex in tank | 301 |
| Dip glass formers in tank | 302 |
| Pick up think latex layer | 303 |
| Adding additional latex layer | 304 |
| Dry latex coating | 305 |
| Vulcanize latex | 306 |

What is claimed is:

1. A male condom comprising:
a closed end covering glans of a penis;
the closed end having a thick latex layer;
a sheath body covering shaft of the penis;
the sheath body having a thin latex layer; and
an open end having a collar to reduce slipping of the condom during intercourse.

2. The condom of claim 1, is made up of latex.

3. The condom of claim 1, wherein the thick latex layer has a thickness of 0.15 mm and length of 85 mm.

4. The condom of claim 1, wherein the thin latex layer has a thickness of 0.05 mm and length of 110 mm.

5. The condom of claim 1, is made up from a dipping process.

6. The condom of claim 1, increases sensation underside of the shaft of the penis.

7. The condom of claim 1, the closed end has an internal diameter in the range 37-39 mm.

8. The condom of claim 1, wherein the sheath body has an internal diameter in the range 32 mm-35 mm.

9. The condom of claim 1, further comprising a throat section at a length of 45 mm from the closed end.

10. A male condom comprising:
a thick latex layer to cover head of a penis;
wherein the thick latex layer reduces the sensation at the underside of the glans;
a thin latex layer to cover the shaft of the penis;
wherein the thin latex layer increases the sensation at the underside of the shaft; and
an open end having a collar to reduce slipping of the condom during intercourse.

11. The condom of claim 11, wherein the thick latex layer has a thickness of 0.15 mm and length of 85 mm.

12. The condom of claim 11, wherein the thin latex layer has a thickness in the range 0.02 mm-0.05 mm and length in the range 100 mm-110 mm.

13. The condom of claim 11, rolls up at the open end after a dipping process during manufacturing.

14. The condom of claim 11, is free from any suspending agent such as benzocaine to delay the ejaculation.

15. The condom of claim 11, is 195 mm in length.

16. A manufacturing process for producing condom comprising the steps of:
dipping glass formers in temperature-controlled tanks,
feeding a latex compound into the temperature-controlled tanks;
picking up thin form of latex by glass formers;
adding more layers of latex to make thick layers of condom;
drying the latex coating; and
vulcanizing the latex to form the condom.

17. The temperature-controlled tanks of claim 16, having temperature in the range 10° C. and 30° C.

18. The process of claim 16, comprises two additional layers of latex at a closed end of the condom.

19. The latex of claim 18, can be one of a synthetic latex or a natural rubber latex.
